(19) United States
(12) Patent Application Publication
(10) Pub. No.: US 2010/0174377 A1
(43) Pub. Date: Jul. 8, 2010

(54) RETICULATED PARTICLE POROUS COATING FOR MEDICAL IMPLANT USE

(75) Inventor: Daniel A. Heuer, Memphis, TN (US)

Correspondence Address: DIANA HOUSTON;SMITH & NEPHEW, INC., 1450 BROOKS ROAD, MEMPHIS, TN, 38116, US

(73) Assignee: SMITH & NEPHEW, INC., Memphis, TN (US)

(21) Appl. No.: 12/602,754

(22) PCT Filed: May 20, 2008
(86) PCT No.: PCT/US08/64242

§ 371 Date: Dec. 2, 2009

Related U.S. Application Data
(60) Provisional application No. 60/942,523, filed on Jun. 7, 2007.

(51) Int. Cl. A61F 2/38; C12N 5/00; B27N 3/00; B32B 37/00; B29C 65/00; B23K 31/02; B22F 3/00; B22F 3/10; A61F 2/28; A61F 2/32

(52) U.S. Cl. 623/20.14; 435/395; 435/396; 264/109; 156/77; 228/101; 419/61; 419/1; 623/16.11; 623/22.11

(57) ABSTRACT

A composition, a medical implant constructed from the composition, and a method of making the composition are described. The composition comprises a porous-coated substrate, the porous coating comprising a reticulated particle coating, the coating being formed by fusing the reticulated particle to the surface, preferably by sintering.

### CROSS-REFERENCE TO RELATED APPLICATIONS

This application claims priority to U.S. provisional application Ser. No. 60/942,523, filed Jun. 7, 2007.

### TECHNICAL FIELD

This invention relates to a new porous structure comprising a sintered reticulated particle porous coating. The new structure is useful in any application where a porous structure is useful, but would be particularly useful as a part of a medical implant material that would promote tissue ingrowth into the implant.

### BACKGROUND OF THE INVENTION

Traditional tissue ingrowth technologies have been relatively successful in helping to restore form and function in various medical implant applications. However, there are some patients, conditions, or situations in which they are not an ideal solution. Traditional technologies have tended to have relatively low porosity, low long-term strength, high stiffness, poor initial stability, or other issues which limit them from being ideal for the broad range of desired applications.

There is particularly an on-going need for improved bone ingrowth structures to serve as a scaffold for bone growth or as a mechanism of attachment for implantable medical devices. It is desirable that such structures provide a porous framework allowing for vascularization as well as new bone ingrowth, and one which provides a compatible site for osteoprogenitor cells and bone growth-inducing factors. The voids and interstices of a porous structure provides surfaces for bone ingrowth, thereby enabling skeletal fixation for permanent implants used for the repair or replacement of bone tissue or in joint replacement applications. The implants may be conventional total joint replacements, such as total hip arthroplasty, total knee arthroplasty, etc., or partial joint replacements, such as hip hemi-arthroplasty. A number of characteristics are known in the art to be important for a successful bone ingrowth structure. These include porosity, biological compatibility, intimate contact with the surrounding bone, and adequate early stability allowing for bone ingrowth. The ideal ingrowth structure should have good strength and ductility, and a stiffness comparable to that of bone. The technology should also ideally be amenable to the easy manufacture of implants of precise dimensions, and permit the fabrication of either thick stand-alone bulk forms or thin coatings attached to solid implant substrates.

One important requirement for successful ingrowth is that the implant material be placed next to healthy bone. An osteoconductive, or bone-growth promoting, porous structure will support the ingrowth of bone tissue when it is placed in physical contact with healthy bone. Proximity to healthy bone allows for the infiltration of bone-forming cells and blood vessels, which are necessary for bone ingrowth.

There have been numerous efforts to develop and manufacture synthetic porous implants having the proper physical properties required to promote bone ingrowth. Implants with porous surfaces of metallic, ceramic, polymeric, or composite materials have been studied extensively over the last two decades.

The use of sintered beads on the surface of medical implants to provide surface porosity and promote bone ingrowth is known (U.S. Pat. No. 3,855,638). However, these techniques result in device with relatively low porosity (<40%) and a relatively smooth outer surface which results in a “poor bite” with adjacent bone. While adequate for some implant applications, these properties do not provide an optimal solution for many of the more challenging ingrowth applications.

Earlier efforts also included the use of fiber metal mesh compositions (U.S. Pat. No. 3,906,550). Although it can produce greater porosity (˜50%), it is still lower than is desirable. Fiber metal mesh also has a relatively smooth outer surface which results in a “poor bite” with adjacent bone. Again, the resulting ingrowth performance is not as great as that desired for many of the more challenging ingrowth applications.

Additionally, plasma-sprayed titanium medical implants have been used (U.S. Pat. No. 3,605,123). These suffer from very low porosity and relatively low attachment strength. As porosity and attachment strength are important characteristics for medical implants, this technology is not thought to be optimal for porous ingrowth applications.

Sintered asymmetric powder compositions have also been used (U.S. Pat. No. 4,206,516). While these exhibit moderate porosity (approximately 60%), they suffer from a lower attachment strength than sintered beads, which may be a disadvantage in some medical implant applications.

Sacrificial Second Phase Compositions, such as Cancellous-Structured Titanium™ and Void Metal Composites (U.S. Pat. No. 3,852,045), have also been used to address the need for a porous framework allowing for revascularization as well as new bone growth. These technologies require complicated manufacturing processes and suffer from relatively smooth outer surfaces resulting in “poor bite” with adjacent bone. These also suffer from relatively low attachment strength.

Integrally cast porous structures have also been used (U.S. Pat. No. 4,781,721). In these compositions, the porous surface is cast simultaneously with the substrate. A resulting advantage is the lack of an abrupt interface (i.e., attachment problems are minimized because the process is not a deposition process). These compositions tend to have larger than desirable structural features and pores, and can only be made from materials that are compatible with the casting process used.

Techniques of selective laser sintering have been used to create a porous framework on a medical implant. However, these techniques have proven to be prohibitively expensive, and are difficult to use to create fine structures.

Several methods of metalizing a reticulated scaffold have been used for medical implant applications, but these approaches tend to be relatively expensive, result in a composition having relatively large pores and relatively low specific surface area, or are difficult to attach to non-planar surfaces. One such technique uses chemical vapor deposition to apply tantalum to the structural members of a reticulated vitreous carbon skeleton (U.S. Pat. No. 5,282,861). This process is very expensive and involves hazardous chemicals, making it a less-than-desirable option. Furthermore, the structure produced is difficult to attach to a solid implant surface, limiting it from being used in a wider variety of applications.

There exists room for improvement in the development of a porous tissue ingrowth structure. It is desirable to have a porous tissue ingrowth structure with more ideal morphological and mechanical characteristics than what is currently available that is easy and relatively inexpensive to produce, and that is applicable to a wide variety of tissue ingrowth applications.

### BRIEF SUMMARY OF THE INVENTION

The present invention is directed to a compositions, medical implants formed of the compositions, and processes for same. The compositions comprise a porous-coated substrate, the porous coating comprising a reticulated particle coating, the coating being formed by fusing the reticulated particle to the surface, preferably by sintering.

In certain embodiments of the invention, there is a porous reticulated structure for cell and tissue ingrowth, the structure comprising fused, distinct three-dimensionally reticulated elements that make up a single continuous composition.

In certain embodiments, each of the reticulated elements comprise no more than one distinct unit cell.

In certain embodiments, the reticulated elements have no distinct unit cells.

In certain embodiments, the porous structure comprises pores having pore sizes of between 50 and 1000 μm.

In certain embodiments, the porous structure comprises pores having pore sizes of between 100 and 500 μm.

In certain embodiments, the reticulated elements comprise a material selected from the group consisting of metal, ceramic, glass, glass-ceramic, polymer, composite, or any combination thereof.

In certain embodiments, the reticulated elements comprise a material selected from the group consisting of titanium, titanium alloy, zirconium, zirconium alloy, niobium, niobium alloy, tantalum, tantalum alloy, cobalt-chromium-molybdenum alloy, or any combination thereof.

In certain embodiments, the porous structure further comprises a solid substrate.

In certain embodiments having a solid substrate, the solid substrate comprises a material selected from the group consisting of a metal, a ceramic, and any combination thereof.

In certain embodiments having a solid substrate, the porous structure covers at least a portion of the surface of the solid substrate and the porous structure and the solid substrate form at least a portion of an implantable medical implant.

In certain embodiments of the implantable medical implant, the implantable medical implant is an orthopaedic implant.

In certain embodiments of the orthopaedic implant, the orthopaedic implant is a hip implant or a knee implant.

In another embodiment, there is a method for producing a porous structure for cell and tissue ingrowth comprising the steps of arranging a plurality of three-dimensionally reticulated particles into a shape, and, fusing the reticulated particles at points where one or more of the particles contact one or more other of the particles to form a single continuous composition.

In certain embodiments, the reticulated particles comprise no more than one distinct unit cell.

In certain embodiments, the reticulated particles have no distinct unit cells.

In certain embodiments, the reticulated particles have a fenestration diameter of between 50 and 1000 μm.

In certain embodiments, the reticulated particles have a fenestration diameter of between 100 and 500 μm.

In certain embodiments, the reticulated particles comprise a material selected from the group consisting of metal, ceramic, glass, glass-ceramic, polymer, composite, and any combination thereof.

In certain embodiments, the reticulated particles consist of a material selected from the group consisting of titanium, titanium alloy, zirconium, zirconium alloy, niobium, niobium alloy, tantalum, tantalum alloy, cobalt-chromium-molybdenum alloy, and any combination thereof.

In certain embodiments, the step of fusing the reticulated particles comprises fusing the reticulated particles with a techniques selected from the group consisting of gluing, sintering, brazing, melting, welding, and any combination thereof.

In certain embodiments, the step of fusing said reticulated particles comprises sintering said reticulated particles.

In certain embodiments, the method further comprises the step of fusing said reticulated particles to a solid substrate.

In certain embodiments wherein the reticulated particles are fused to a solid substrate, the method further comprises the step of forming an implantable medical implant from the fused reticulated particles and solid substrate.

In certain embodiments, the step of forming an implantable medical implant comprises forming a hip implant or a knee implant.

In another embodiment of the invention, there is a process for producing three-dimensionally reticulated particles with no more than one unit cell comprising the steps of: providing a three-dimensionally reticulated bulk structure; segmenting the bulk structure to produce discrete reticulated particles; and, separating the discrete reticulated particles by size based on an original unit cell diameter of the bulk structure.

In certain embodiments, the process further comprises the step of embrittling said bulk structure prior to said step of segmenting.

In certain embodiments, the step of embrittling is accomplished through cryogenic processing.

In certain embodiments, the step of embrittling is accomplished through a reversible chemical reaction.

In certain embodiments, the reversible chemical reaction is a hydride/dehydride process.

In certain embodiments, the step of segmenting said bulk structure comprises crushing said bulk structure.

In certain embodiments, the three-dimensionally reticulated bulk structure comprises scrap from a bulk reticulated structure.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated that the conception and specific embodiment disclosed may be readily utilized as a basis for modifying or designing other structures for carrying out the same purposes of the present invention. It should also be realized that such equivalent constructions do not depart from the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1 is a schematic illustration of a bulk porous tissue ingrowth structure created by the sintering together of one or more layers of reticulated metal particles.

FIG. 2 is a schematic illustration of the porous tissue ingrowth coating created on a solid implant surface through the sintering of one or more layers of reticulated metal particles.

FIG. 3 is a schematic illustration of one method of segmenting a bulk reticulated structure into reticulated particles by crushing a material such as a reticulated metal or ceramic foam.

FIG. 4 is a schematic illustration of a single unit cell of a three-dimensionally reticulated structure.

FIG. 5 is a schematic illustration of struts and nodes is portions of reticulated elements.

FIG. 6 is a schematic illustration of the deficiencies of the prior art with respect to non-planar surfaces which are overcome by the present invention.

FIG. 7 compares a structure comprising distinct reticulated particles to the original continuous reticulated bulk structure from which the particles were produced.

### DETAILED DESCRIPTION OF THE INVENTION

As used herein, “a” and “an” include both the singular and the plural and mean one or more than one. In general herein, the singular encompasses the plural and the plural encompasses the singular unless otherwise indicated or is evident from the context.

As used herein, the term “reticulated structure” means a structure having an interconnected network of open-cells defined by a continuous array of struts and nodes. A reticulated structure can generally be described as having an open-celled foam or sponge-like form.

As used herein, the term “strut” means a material boundary between fenestrations in an open-celled reticulated structure.

As used herein, the term “node” means the location of the intersection of a plurality of struts in an open-celled reticulated structure.

As used herein, the term “terminal strut” means a strut that is bound to only one node. In a typical bulk reticulated structure, terminal struts only occur at the surface of the bulk structure where the structure has been sectioned.

As used herein, the term “terminal node” means a node from which struts only emanate on one side. In a typical bulk reticulated structure, terminal nodes only occur at the surface of the bulk structure where the structure has been sectioned.

As used herein, the term “fenestration” means the generally circular opening connecting two unit cells of an open-celled reticulated structure defined by a polygonal (typically pentagonal or hexagonal) arrangement of struts and nodes.

As used herein, the term “unit cell” means the generally spherical void space in a reticulated structure defined by a polyhedral (typically dodecahedral) arrangement of struts and nodes.

As used herein, the term “distinct unit cell” means a continuous array of struts and nodes that makes up at least half of a polyhedron that would constitute a unit cell for that structure.

As used herein, the term “reticulated element” means a morphologically distinct three-dimensional strut-and-node-type structure comprised of 1) at least one node and at least three struts, the axes of which do not all fall within the same plane, or 2) at least two nodes and at least three struts. A reticulated element is distinguished by the presence of terminal struts or terminal nodes which define its extent. A reticulated element may or may not be part of a larger continuous structure, in which the volume defined by the extent of each element may overlap.

As used herein, the term “reticulated particle” means a reticulated element which is not a part of a larger continuous structure.

As used herein, the term “fusing” or “fusion” or the expression “to fuse” means the joining of two distinct aggregates into a materially continuous unitary whole. “Materially continuous” means connected by a material interaction and not merely connected by physical contact; i.e., not a mechanical joining such as that resulting from materials strands which are intertwined with other material strands. This can be accomplished by any means, including, but not limited to, gluing, sintering, brazing, melting, welding, etc., and other means in which aggregates are joined by a material interaction and not merely a mechanical interaction.

In some embodiments of the invention, disadvantages of the known art described above are overcome or ameliorated. In other embodiments, there is provided a process for easily producing a bulk reticulated structure in any shape. Another embodiment of the invention is to provide a process for simultaneously forming and attaching reticulated structures on contoured solid surfaces. In other embodiments, there is provided a process for producing a reticulated structure which is substantially open and interconnected and that can have a smaller pore size than is possible with the known art.

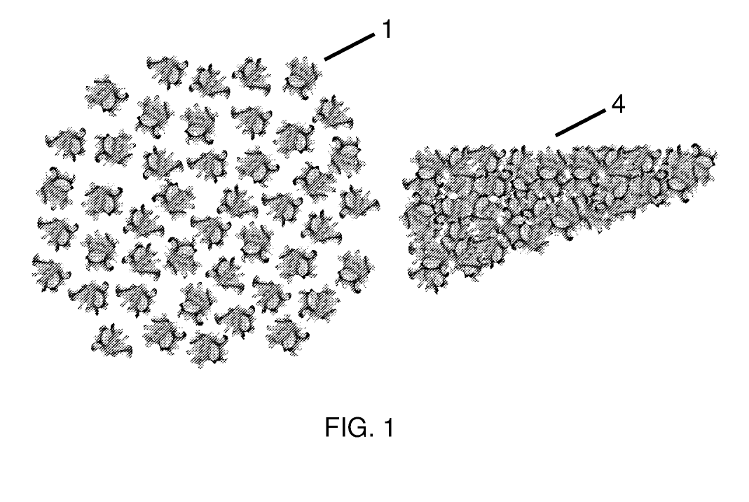

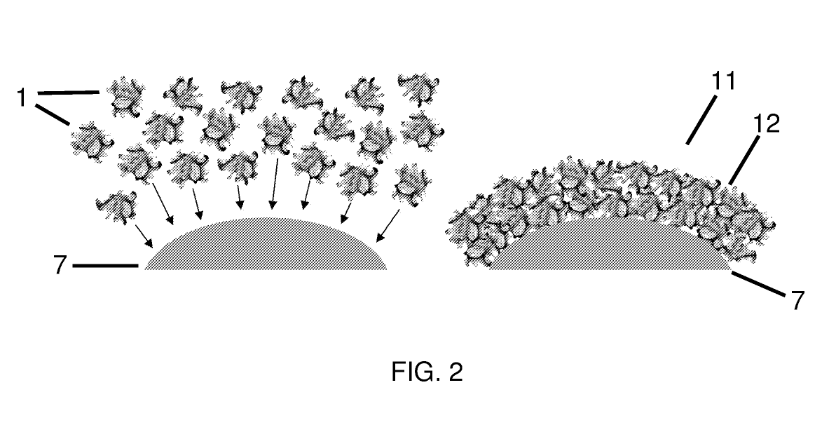

The present invention relates to a porous tissue ingrowth structure, preferably for use in a medical implant application, created by the fusing together of one or more layers of reticulated particles. A schematic illustration showing a stand-alone bulk porous tissue ingrowth structure created by the fusing together of one or more layers of reticulated particles is shown in FIG. 1. A schematic illustration showing the porous tissue ingrowth structure created on the surface of a solid substrate by the fusing together of one or more layers of reticulated particles to one another and to the solid substrate is shown in FIG. 2.

In one illustrative embodiment shown in FIG. 1, reticulated metal particles 1 are formed into a shape and sintered to bond the particles at points of contact with other particles, forming a single continuous porous composition; in the example illustrated in FIG. 1, a wedge-shaped composition, 4.

In another embodiment, shown in FIG. 2, reticulated metal particles 1 are applied to the surface of a solid metal substrate 7 and sintered to bond particles to other particles and to the surface at respective points of contact, forming a final product 11 comprising a single continuous porous composition 12 attached to the surface of the solid substrate 7.

The reticulated particles that are used to produce the final composition and device can be made up of any material or materials and be formed by any process known in the art. Preferably, this is accomplished by segmentation of a reticulated metal or ceramic foam. Alternatively, this may be accomplished by segmentation of a reticulated precursor to a metal or ceramic foam, such as a metal or ceramic powder-filled reticulated polymer foam that is further processed into reticulated particles of a composition derived from the powdered filler material. Alternatively, this may be accomplished by segmentation of a first reticulated scaffold composition that is subsequently coated with a second coating composition. This coating could be applied by chemical vapor deposition, physical vapor deposition, powder-coating, slurry-coating, sol-gel coating, electroplating, or other suitable coating method.

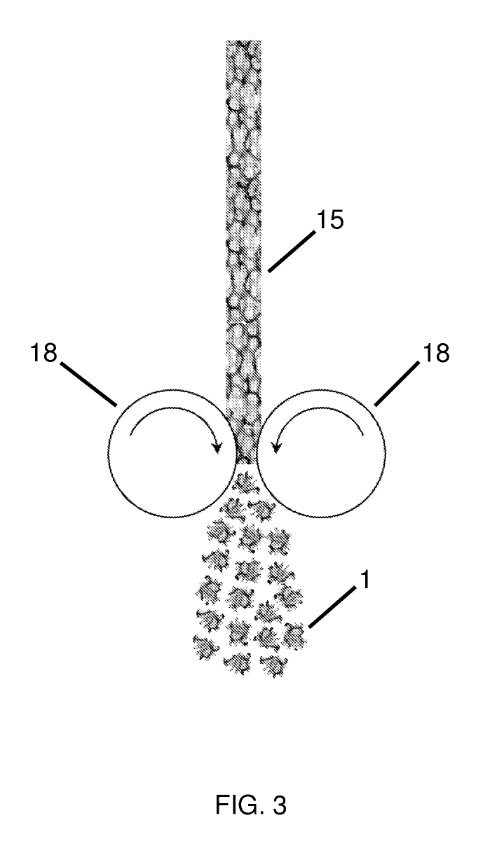

Segmentation may be accomplished by any process know in the art. A schematic illustration of one example of this is shown in FIG. 3. Preferably, this is accomplished by crushing material 15 (which may be, by way of non-limiting examples, a reticulated metal or ceramic foam) using one or more crushing rollers 18 to produce reticulated particles 1. In one embodiment, the production of reticulated ceramic particles of an ideal size range is accomplished by crushing of a reticulated ceramic foam between a set of rollers in a specific orientation (with or without the assistance of a conveyor or roller of feeding system to advance the foam through the set of crushing rollers). This is shown schematically in FIG. 3. Alternatively, this may be accomplished by grinding, chopping, or cutting the material. Other methods of segmentation may include the application of sonic energy to a reticulated structure and/or the controlled detonation of a reticulated structure. It is envisioned that segmentation applicable to the invention herein may also be accomplished by segmentation methods and processes to be later developed.

To be made more amenable to segmentation, a foam made from a ductile material can be made temporarily more brittle through a reversible process prior to segmentation. For example, a ductile titanium foam can be hydrided to be made more brittle prior to segmentation. This can then be followed by dehydriding during the sintering of the structure (or during a separate dehydriding step prior to the sintering of the structure) to regain the ductile attributes of the original titanium foam Likewise, a foam that is ductile or resilient at room temperature may also be made temporarily more brittle by exposure to very low temperatures such as by exposure to a cryogenic composition prior to segmentation. This can then be followed by returning the structure to room temperature to regain the ductile or resilient attributes of the original foam.

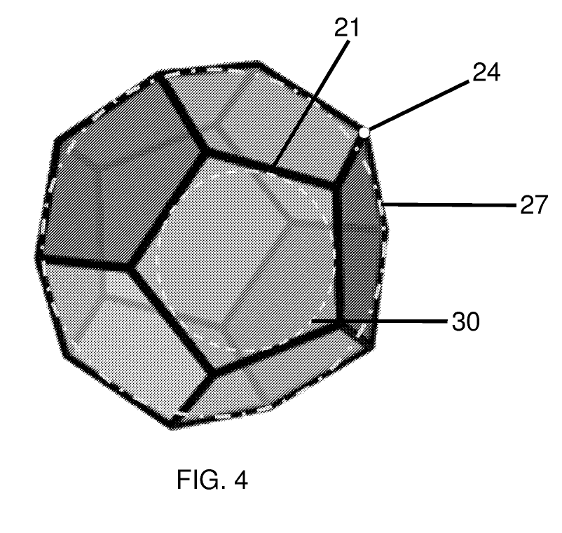

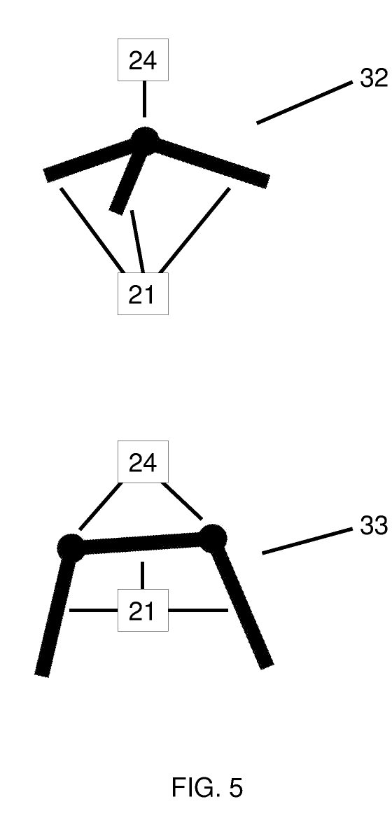

Reticulated open-celled bulk structures consist of an arrangement of struts connected by nodes where three or more of these struts meet. This structure is shown schematically in FIG. 4. The void space in such a structure consists of roughly spherical polyhedral unit cells 27 which are connected to one another through open windows, or fenestrations, 30, typically formed by 5 to 7 (or other number of) struts falling within the same plane. A strut 21 forms the border between fenestrations, while a node 24 is where a plurality of struts intersect. A further schematic illustration of “struts” and “nodes” is provided in FIG. 5, showing struts 21 and nodes 24 in portions of reticulated elements 32 and 33. The exemplary fenestration 30 shown in FIG. 4 is pentagonal, but can be considered to be approximately “circular”. In this way, the “diameter” of such fenestrations is measured from one strut through the fenestration to an opposite (i.e., non-adjacent) strut. The pore size of reticulated open-celled bulk structures are characterized by both the diameter of the unit cell and the diameter of the fenestrations. With many of the technologies known in the art, there are financial or technical challenges in producing a reticulated open-celled bulk structures with a sufficiently small pore size to be considered ideal for tissue ingrowth. Furthermore, to this end, as far as the inventors are aware, porous reticulated structures comprising fused, distinct three-dimensionally reticulated elements that make up a single continuous composition are not known. Segmenting a reticulated open-celled bulk structure with a larger pore size, however, reduces or eliminates the number of larger diameter unit cells, producing particles in which the pore size is dominated by the diameter of the smaller fenestrations. One object of this invention is to enable the use of less expensive and easier to manufacture reticulated structures with larger unit cells to produce final structures with a pore size within the desired range.

Any method of making metallic or ceramic reticulated foams or structures are applicable in the present invention. Several methods have previously been utilized to make open-celled reticulated structures. In one such general method, a sinterable powder is mixed with a foamable resin or resin system. Upon foaming, the surface tension in the resin forces the powder into the strut and node regions of the foam, with thin resin windows separating the unit cells of the foam. The resulting closed-cell reticulated structure is then heated to volatilize or burn out the resin and sinter the remaining powder into an open-celled reticulated structure (U.S. Pat. Nos. 1,919,730; 2,917,384; 3,078,552; 3,833,386; 4,569,821; 5,171,720; 5,213,612; 5,976,454; and 6,087,024). In another method, one open-celled reticulated structure is used to create an investment casting to form an identical structure in a different material. In this method, a negative mold is made around the structural features of the starting structure and the starting structure is destructively removed, usually by combustion, volatilization, melting, or other means. A fluid material is then injected into the vacated cavity and solidified, and the negative mold is destructively removed leaving a final open-celled reticulated structure with a chemistry derived from that of the fluid material (U.S. Pat. Nos. 3,616,841; 3,946,039; 4,235,277; 4,600,546; and 4,781,721). In another group of methods, an open-celled reticulated structure is used as a scaffold. In one such method, the scaffold is infiltrated with a slurry containing a sinterable powder. The excess slurry is then removed leaving a uniform thin coating over all of the internal structural elements of the scaffold. The structure is then heated to sinter the coating, creating an open-celled reticulated structure with a chemistry derived from the sinterable powder material (U.S. Pat. Nos. 3,090,094; 3,097,930; 3,111,396; 3,408,180; 4,004,933; 4,024,212; 4,056,586; 4,371,484; 4,517,069; 4,803,025; 4,866,011; 5,531,955; 5,839,049; 6,387,149; 6,840,978; and 6,977,095). In some variations of this process, the original scaffold is removed during sintering, and in others the scaffold remains in the final product (U.S. Pat. No. 5,185,297). In some variations of this process, the slurry-coating step is replaced by coating the structure first with an adhesive, and then with a dry sinterable powder (U.S. Pat. Nos. 5,531,955; 5,881,353; and 6,706,239). The foregoing are merely illustrative and non-limiting examples of commonly-known methods to make a reticulated structure which is useful as a source of reticulated metal particles to make the new porous reticulated structure of the present invention. It is expected other methods and resulting reticulated structures would also be applicable, including any methods and resulting reticulated structures that are yet to be developed.

Reticulated particles can be formed as such, or can be created by the segmentation of a larger bulk reticulated structure. In certain circumstances, reticulated particles can be created by the segmentation of otherwise unusable or waste material, such as that removed during the shaping of bulk reticulated structures, bulk reticulated structures that do not meet dimensional tolerances, etc. This otherwise unusable material is typically discarded or treated as scrap material with little to no value, possibly even incurring cost in the form of special storage requirements or disposal fees. The ability to recycle otherwise unusable or waste material could represent a substantial cost savings.

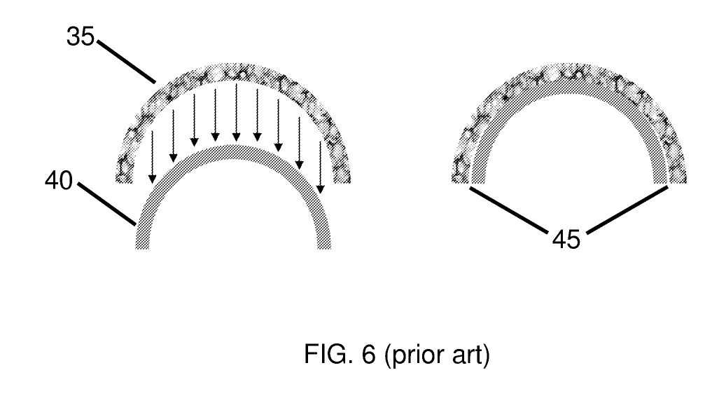

It is difficult to create a high-strength biocompatible reticulated metallic foam with the desired porosity, pore size, and surface area and which can be easily applied to a wide range of implant applications using currently available technologies. Existing technologies do not produce structures with sufficient strength, are too expensive to be economically feasible, are limited to producing structures with a pore size larger than is thought to be ideal for bone ingrowth, or are technologically limited only to certain ingrowth applications. Some bulk metallic foams can be made with a desirable structure and strength, but have been found to be very difficult to attach to a solid implant substrate, especially where the implant surfaces are non-planar. This has led to the development of complicated attachment procedures (U.S. Patent Application Publication No. 2005/0184134). Using the process of the present invention, relatively low-cost, high-strength reticulated metallic structures or coatings can easily be created in any shape or on any surface with an ideal porosity, pore size, and surface area. This advantage of the present invention can be understood by reference to the prior art illustrated in FIG. 6. As illustrated in FIG. 6, attaching a formed porous structure 35 to a non-planar surface using the methods of the prior art tends to result in a less-than-optimal fit as shown by gaps in contact 45. By sintering the material to the surface of the device to be coated, the difficulties in attaching a foam material are overcome and contact in the final device is improved.

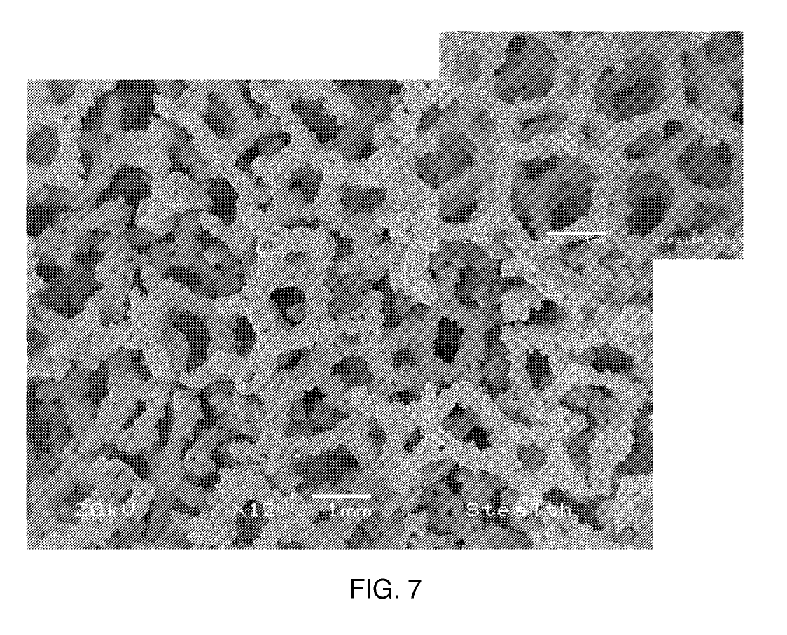

Additionally, the resulting porous layer has structural advantages over that which is created when a bulk foam material is attached to a surface to create a porous surface. The sintering of reticulated particles onto a surface to create a porous surface results in a surface having many small irregular-shaped cells. FIG. 7 compares the structure of the fused reticulated particles of the invention (large image) to those of the original bulk reticulated structure prior to segmentation (upper right image). The resulting surface will exhibit better performance in medical implant applications where bone and tissue ingrowth is the primary goal.

The flexibility of this technology comes from the reticulated particles, which can be easily made into any bulk form or applied to any surface (similar to other powder metallurgy techniques), yet has greater porosity and pore size than is created using solid metal powder particles. The strength of the structure is also enhanced over that of the original metallic foam due to the increased density and increased number of necks created between the particles during sintering.

Another advantage is that the final structure has a more textured surface than typical bulk reticulated structures. Most bulk reticulated metallic structures need to be shaped with wire electrical discharge machining (EDM), which produces a relatively smooth surface. This is because traditional machining results in smearing of the metal which closes surface pores. Because the present concept uses individual reticulated particles, the coatings can be applied uniformly while producing a rough surface with more optimal frictional properties.

While sintering of the reticulated particles onto the surface is the preferred method of fusing the particles to each other and to the surface, other methods are applicable and within the scope of the present invention. For example, in some embodiments, the reticulated particles may be a polymer or a polymer composite (a composite material comprising at least one polymer and at least one non-polymer). In such cases, fusing may be accomplished by partial dissolution of the particle composition in a chemical solvent by removal of the solvent and fusing the particles to each other and to the surface. Although a polymer or a polymer composite is provided as an illustrative example, it is possible that other materials that have some solubility in a chemical solvent may also be used.

A minimum pore size of about 50 μm is generally thought to be necessary to obtain mineralized bone ingrowth. Pore sizes up to 1000 μm are preferred. Pore sizes greater than 1000 μm are still useful in the present invention and are within its scope, but such large sizes are less preferred. Therefore, a pore size (or fenestration diameter) of between 50 and 1000 μm is preferred. Ideal bone ingrowth is believed to be obtained in structures with pore sizes ranging from 100 to 500 μm. Therefore, a pore size (or fenestration diameter) of between 100 and 500 μm is even more preferred. In some embodiments, the reticulated particles comprise a material selected from the group consisting of metal, ceramic, glass, glass-ceramic, polymer, composite, or any combination thereof. In some embodiments, the reticulated particles comprise a material selected from the group consisting of titanium, titanium alloy, zirconium, zirconium alloy, niobium, niobium alloy, tantalum, tantalum alloy, cobalt-chromium-molybdenum alloy, or any combination thereof.

The resulting composition is an exceptional biomaterial that, when placed next to bone or tissue, initially serves as a prosthesis and then functions as a scaffold for regeneration of normal tissues. It satisfies the need for an implant modality that has a precisely controllable shape and at the same time provides an optimal matrix for cell and tissue ingrowth. Additionally, the physical and mechanical properties of the porous structure can be specifically tailored to the particular application at hand. This new implant offers the potential for use in orthopaedic applications, particularly for use in orthopaedic implants such as, but not limited to, hip and knee implants. As an effective substitute for autografts, it will also reduce the need for surgery to obtain those grafts.

A major advantage of the open cell structure described herein is that it is readily shapeable to nearly any configuration, simple or complex, simply by shaping the substrate material prior to application of the surface material. This facilitates exact contouring of the implant for the specific application and location; precise placement is enhanced and bulk displacement is prevented. Additionally, it appears that any final shaping/trimming needed at surgery can be accomplished on the final device using conventional dental or orthopedic equipment available at the time of surgery.

The optimal conditions for fracture healing and long-term stability can be met if an implant can be made to be motionlessness along all the interfaces necessary for a stable anchorage, thereby excluding (to the greatest extent possible) all outside influences on the remodeling process and allowing the local stress/strain field to control ingrowth.

Following implantation and initial tissue ingrowth, the foam device stays where it is placed without retention aids, a reflection of precise contouring and the rapid ingrowth of fibrovascular tissue to prevent dislodgement. The binding between bone and implant stabilizes the implant and prevents loosening. These implants thus will not need to be held in place by other means (e.g. sutures or cement); rather, the ingrowth of natural bone is encouraged by the nature of the implant itself. Tissue ingrowth would not be a contributing factor to device retention for a period following implantation, however, until a substantial amount of ingrowth had occurred.

For medical implant applications, it is preferable that the reticulated particles used to form the porous surface are formed from biocompatible metals or metal alloys. Non-limiting examples of such biocompatible metals or metal alloys are titanium, titanium alloy, zirconium, zirconium alloy, niobium, niobium alloy, tantalum, tantalum alloy, cobalt-chromium-molybdenum alloy, and any combination thereof. Alternatively, the reticulated particles can be formed from biocompatible ceramics, such as hydroxyapatite, tri-calcium phosphate, bioactive glasses, and any combination thereof. Alternatively, the structure can be composed of a mixture of reticulated particles of different materials or the reticulated particles themselves can be composed of a mixture of different materials.

Although the present invention and its advantages have been described in detail, it should be understood that various changes, substitutions and alterations can be made herein without departing from the invention as defined by the appended claims. Moreover, the scope of the present application is not intended to be limited to the particular embodiments of the process, machine, manufacture, composition of matter, means, methods and steps described in the specification. As one will readily appreciate from the disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

What is claimed is:

1. A porous reticulated structure for cell and tissue ingrowth, said porous reticulated structure comprising a plurality of distinct three-dimensional reticulated elements, each of said reticulated elements being fused to at least one other reticulated element thereby forming a single continuous composition.

2. The porous structure of claim 1, wherein each of said reticulated elements comprise no more than one distinct unit cell.

3. The porous structure of claim 1, wherein said reticulated elements have no distinct unit cells.

4. The porous structure of claim 1, wherein said porous structure comprises pores having pore sizes of between 50 and 1000 μm.

5. The porous structure of claim 1, wherein said porous structure comprises pores having pore sizes of between 100 and 500 μm.

6. The porous structure of claim 1, wherein said reticulated elements comprise a material selected from the group consisting of metal, ceramic, glass, glass-ceramic, polymer, composite, or any combination thereof.

7. The porous structure of claim 1, wherein said reticulated elements comprise a material selected from the group consisting of titanium, titanium alloy, zirconium, zirconium alloy, niobium, niobium alloy, tantalum, tantalum alloy, cobalt-chromium-molybdenum alloy, or any combination thereof.

8. The porous structure of claim 1, further comprising a solid substrate.

9. The porous structure of claim 8, wherein said solid substrate comprises a material selected from the group consisting of a metal, a ceramic, and any combination thereof.

10. The porous structure of claim 8, wherein said porous structure covers at least a portion of the surface of said solid substrate and said porous structure and said solid substrate form at least a portion of an implantable medical implant.

11. The porous structure of claim 10, wherein said implantable medical implant is an orthopaedic implant.

12. The porous structure of claim 11, wherein said orthopaedic implant is a hip implant or a knee implant.

13. A method for producing a porous structure for cell and tissue ingrowth comprising the steps of:
arranging a plurality of three-dimensionally reticulated particles into a shape, and,
fusing said reticulated particles at points where one or more of said particles contact one or more other of said particles to form a single continuous composition.

14. The method of claim 13, wherein said reticulated particles comprise no more than one distinct unit cell.

15. The method of claim 13, wherein said reticulated particles have no distinct unit cells.

16. The method structure of claim 13, wherein said reticulated particles have a fenestration diameter of between 50 and 1000 μm.

17. The method structure of claim 16, wherein said reticulated particles have a fenestration diameter of between 100 and 500 μm.

18. The method of claim 13, wherein said reticulated particles comprise a material selected from the group consisting of metal, ceramic, glass, glass-ceramic, polymer, composite, and any combination thereof.

19. The method of claim 13, wherein said reticulated particles consist of a material selected from the group consisting of titanium, titanium alloy, zirconium, zirconium alloy, niobium, niobium alloy, tantalum, tantalum alloy, cobalt-chromium-molybdenum alloy, and any combination thereof.

20. The method of claim 13, wherein said step of fusing said reticulated particles comprises fusing said reticulated particles with a techniques selected from the group consisting of gluing, sintering, brazing, melting, welding, and any combination thereof.

21. The method of claim 20, wherein said step of fusing said reticulated particles comprises sintering said reticulated particles.

22. The method of claim 13, further comprising the step of fusing said reticulated particles to a solid substrate.

23. The method of claim 22, further comprising the step of forming an implantable medical implant from said fused reticulated particles and solid substrate.

24. The method of claim 23, wherein said step of forming an implantable medical implant comprises forming a hip implant or a knee implant.

25. A process for producing three-dimensionally reticulated particles with no more than one unit cell comprising the steps of:
providing a three-dimensionally reticulated bulk structure;
segmenting said bulk structure to produce discrete reticulated particles; and,
separating said discrete reticulated particles by size based on an original unit cell diameter of said bulk structure.

26. The process of claim 25, further comprising the step of embrittling said bulk structure prior to said step of segmenting.

27. The process of claim 25, wherein said step of embrittling is accomplished through cryogenic processing.

28. The process of claim 25, wherein said step of embrittling is accomplished through a reversible chemical reaction.

29. The process of claim 28, wherein said reversible chemical reaction is a hydride/dehydride process.

30. The process of claim 25, wherein said step of segmenting said bulk structure comprises crushing said bulk structure.

31. The process of claim 25, wherein said three-dimensionally reticulated bulk structure comprises scrap from a bulk reticulated structure.
